(19) United States
(12) Patent Application Publication
(10) Pub. No.: US 2008/0260574 A1
(43) Pub. Date: Oct. 23, 2008

(54) Filtration and Purification Systems

(76) Inventor: Kosta Kapitoures, Queensland (AU)

Correspondence Address: ABELMAN, FRAYNE & SCHWAB, 666 THIRD AVENUE, 10TH FLOOR, NEW YORK, NY, 10017, US

(21) Appl. No.: 11/660,563

(22) PCT Filed: Aug. 23, 2005
(86) PCT No.: PCT/AU2005/001264

§ 371 Date: Sep. 28, 2007

(30) Foreign Application Priority Data
Aug. 31, 2004 (AU) 2004205335

(51) Int. Cl. A61L 9/00; B01D 50/00; F24F 3/16; F25D 23/00; A61L 9/14

(52) U.S. Cl. 422/4; 96/223; 62/78; 62/259.1

(57) ABSTRACT

An air conditioning system (10) including: extraction means for extracting air from a confined space; filtration and purification means for filtering particulate from the extracted air and purifying the air, the filtration and purification means including a separation column (11, 12) having an extracted air inlet (26) for receiving extracted air therethrough, a packed bed (31) at a predetermined first level above the extracted air inlet, water distribution means (32) above the packed bed for distributing cold water onto the packed bed, a clean air outlet (27a) above the packed bed for discharging filtered and purified air to the confined space, and a contaminated water outlet (22a).

### FIELD OF THE INVENTION

This invention relates to filtration and purification systems and also air conditioning systems.

The invention has particular application to the filtration and purification of air in facilities where there is a high level of sensitivity to contaminants, for example, hospitals, and reference will be made to that application. However, it will be appreciated that the invention could have application to other facilities and to other types of atmospheres.

### BACKGROUND

Hospitals typically rely on High Efficiency Particulate Air (HEPA) filters to remove contaminants from air being supplied to sensitive rooms such as operating theatres, intensive care wards and contagious disease wards from an air conditioning system. While such filters may prevent the ingress of a very high percentage of dust, bacteria, viruses and other micro-organisms into the sensitive rooms, they do not purify the air. That is to say, they do not kill entrained micro-organisms, but rather simply provide a barrier to their entry into the excluded rooms and they continue to circulate throughout the air supply system and contaminate other non-excluded rooms unless they are caught in the filters and cannot escape.

The present invention is aimed at providing a filtration and purification system in which undesirable micro-organisms are killed and removed from the air so that air being circulated in a facility or a room in a facility is being continually cleaned and purified. The invention is also aimed at providing a combined air conditioning, filtration and purification system which is reliable and efficient in use.

### SUMMARY OF THE INVENTION

With the foregoing in view, the present invention resides broadly in a filtration and purification system including:

extraction means for extracting air from a confined space;

filtration and purification means for filtering particulate from the extracted air and purifying the air by killing micro-organisms entrained in the extracted air.

In some embodiments, the filtration and purification means may have separate filtration means and purification means whereby micro-organisms can first be filtered from the extracted air and then the purified air can be cleaned. Advantageously, that order provides for the filtration system to remove any residual sanitizing agent from the air prior to it being discharged into the confined space. In some circumstances it may be advantageous to reverse the order. Thus, in another aspect the invention resides broadly in a filtration and purification system including:

extraction means for extracting air from a confined space;

purification means for purifying the air by killing micro-organisms which are entrained in the extracted air; and

filtration means for filtering particulate from the extracted air.

In another aspect the invention resides broadly in an air conditioning system including:

extraction means for extracting air from a confined space;

filtration and purification means for filtering particulate from the extracted air and purifying the air, the filtration and purification means including a separation column having an extracted air inlet for receiving extracted air therethrough, a packed bed at a predetermined first level above the extracted air inlet, water distribution means above the packed bed for distributing cold water onto the packed bed, a clean air outlet above the packed bed for discharging filtered and purified air to the confined space, and a contaminated water outlet.

Preferably, the cold water is sanitized water which is adapted to kill unwanted micro-organisms. It is also preferred that the water distribution means be adapted to provide a continuous spray across the whole upper surface of the packed bed at a predetermined flow rate sufficient to provide a continuous flow therethrough whereby the growth of micro-organisms in the packed bed is prevented. It is also preferred that the sanitized water distributed onto the packed bed be at a temperature of less than about 25 degrees Celsius but preferably less than about 5 degrees Celsius and more preferably less than about 2 degrees Celsius. The lower temperature water is believed to result in better separation of particulate and gaseous pollutants from the extracted air and thus the system provides better quality filtration at lower temperature water. It is also believed that micro-organisms entrained in the extracted air become entrapped in the sanitized liquid droplets and/or are flushed from the extracted air and immersed in the sanitized liquid. Typically, in a high quality filtration system, the cold water would be supplied by a chiller at about 2 degrees Celsius to achieve suitable separation performance (that is, filtration and purification) and be reheated to achieve a suitable room temperature after separation. Thus, in one form, the present invention provides a system in which water which is normally chilled only for air conditioning purposes is also used for filtration purposes. Additionally, the cold water and the packed bed combine to cool the extracted air being returned to the confined space, thus providing a combination air conditioning, filtration and purification system.

If desired, the air conditioning system may have the filtration system separate from the purification system. In such case, the filtration system preferably includes a separation column similar to that previously described. However, the separation column for the filtration system may use unsanitized water for spraying the packed bed and may also includes means for maintaining the temperature of the air above the packed bed at a temperature above about 5 degrees Celsius and preferably above about 20 degrees Celsius to obtain better separation of gaseous pollutants from the extracted air. In such form, the purification system may also carry out a “filtration” process, for example by including a packed bed as previously described thus effectively resulting in a double filtration process as well as an air conditioning process. In such a system, the purification process will preferably be carried out prior to entry of the air to the separation column.

Preferably, the system includes a tank to which the contaminated water flows from the contaminated water outlet and control means are provided for maintaining the level of sanitizing agent in the water at a desired predetermined level. In such form of the invention, it is preferred that the control means also controls the removal of contaminated water from the tank and the addition of fresh water thereto in order to maintain the level of contaminants in the water below a predetermined maximum desired level. Additionally, the system preferably includes a separate filtration system for filtering contaminants from the contaminated water. Typically such system would include a replaceable cartridge filter of known type.

Preferably, the filtration and purification means is remote from the confined space, for example in the case of a hospital, in a plant room separate from the wards and theatres. Advantageously, such an arrangement results in micro-organisms being removed from the sensitive areas prior to the air being purified.

In another aspect the invention resides broadly in a method of sanitizing a room or a building, including:

continuously extracting air from the room or building;

passing the extracted air through filtration and purification means thereby filtering particulate from the extracted air and purifying the air by entrapping micro-organisms entrained therein in a sanitized liquid; and

returning at least some of the extracted air to the room or building after filtration and purification by the filtration and purification means.

Preferably, the filtration and purification means includes a separation column as previously described. However, in a much preferred form, the filtration and purification means includes two separation columns.

Preferably, the method includes passing the air through a second separation column downstream of the first separation column and spraying unsanitized water onto the packed bed of that separation column.

### BRIEF DESCRIPTION OF THE DRAWINGS

In order that the invention may be more easily understood and put into practical effect, a preferred embodiment of the invention will now be described with reference to the accompanying drawings wherein:

FIG. 1 is a schematic elevation of a filtration and purification system according to the present invention connected to the air conditioning system of a building; and

FIG. 2 is a schematic plan view of another filtration and purification system according to the present invention.

### DETAILED DESCRIPTION OF THE DRAWINGS

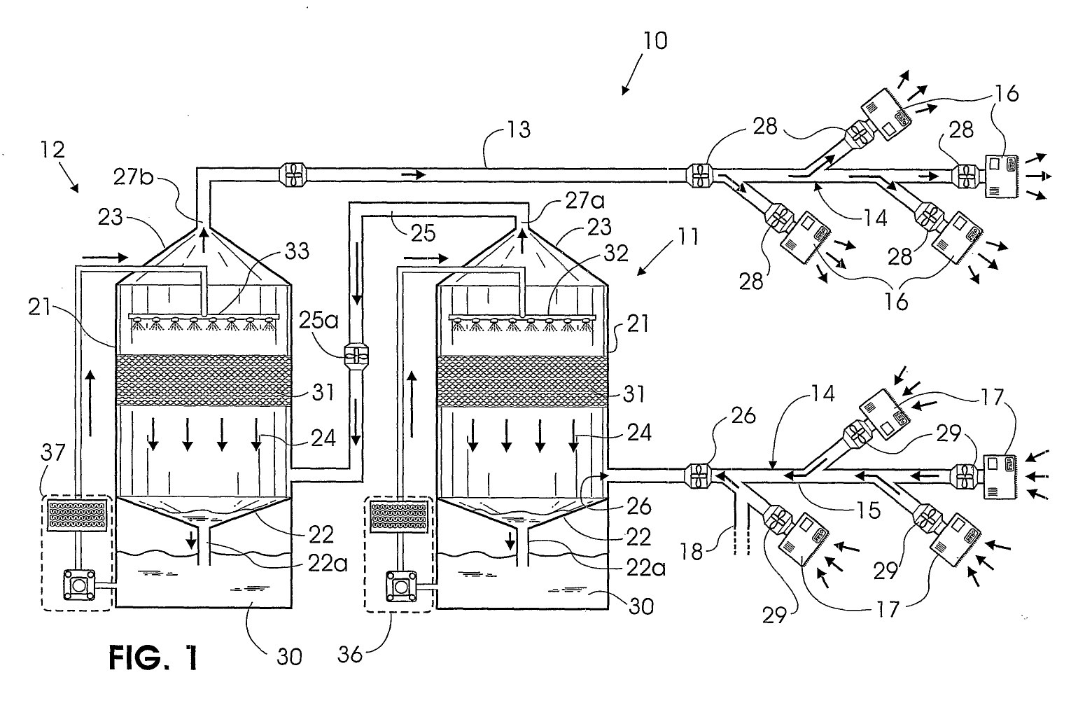

The filtration, purification and air conditioning system 10 illustrated in FIG. 1 includes a first separation column 11 which is connected to a second separation column 12 which in turn is connected to the supply air duct 13 of an air conditioning type duct system 14 of a doctor's surgery with the return air duct 15 returning polluted air from the surgery to the separation column and the supply air system duct supplying filtered and purified air to the surgery from the second separation column. The filtered and purified air enters the surgery through supply air registers shown typically at 16 while the polluted air leaves the surgery through return air grills shown typically at 17. Fresh air is taken into the system via a fresh air inlet grill shown typically at 18.

The separation columns 11 and 12 each include a housing comprising a generally cylindrical main body 21, a conical base 22 depending from the main body and a conical top 23 contiguous with the main body and which together with the main body and the base define a chamber 24 in which separation of gaseous and particulate pollutants from the return air extracted from the surgery can take place. A polluted air inlet 26 is provided in the lower part of the main body to which the return air duct is connected for passage of the polluted air from the surgery to the separation column. Towards the upper end of the first separation column, a purified air outlet 27a is provided in the main body to which a transfer duct 25 is connected for transfer of air to the inlet of the second column via an in-line fan 25a. Similarly, a purified and filtered air outlet 27b is provided in the main body of the second column towards its upper end to which the supply air duct 28 is connected for supply of purified and filtered air to the surgery. For that purpose, a plurality of supply air fans 28 are mounted in the supply air duct, although in other systems, a supply air fan could be housed in a plant room beside the separation column. Similarly, return air fans 29 assist in extracting the air from the surgery but they also could be replaced by a suitable return air fan housed in the plant room. A contaminated water outlet 22a is provided at the apex of the conical base in each column for discharging contaminated water to a tank 30 below the separation chamber.

A packed bed 31 is supported in the upper part of each separation chamber above the polluted air inlet 26. Suitably, the bed is packed with expanded metal foil of the type described in the international application No. PCT/AU01/01028 by the present inventor which is incorporated herein by way of reference. Other forms of expanded foil beds may be used although perhaps to lesser effect.

A sprinkler system 32 is supported in the separation chamber of the first column above the packed bed and is adapted to sprinkle chilled water at about 2° C. onto the packed bed so that it can flow down through the voids in the metal foil under the force of gravity. A sprinkler system 33 is similarly supported in the separation chamber of the second column and is arranged to ensure that chilled sanitized water is distributed wholly across the upper face of the packed bed. Suitably, the sprinkler system of the first separation column sprinkles water at a flow rate which is sufficient to prevent the growth of micro-organisms in the packed bed as it flows therethrough. That flow rate will depend on the level of sanitization in the water, the type of micro-organisms in the polluted air and various other factors including temperature.

In use, chilled water is supplied to the sprinkler systems 32 and 33 by respective pump/chiller plants 36 and 37 which are of the type typically used for chilling water for air conditioning systems, through pipe systems 38 and 39 respectively. The chilled water flows through the packed bed in each column while the extracted air flows upwards through the packed bed where gaseous pollutants and particulate pollutants (including micro-organisms) are entrapped in water droplets flowing therethrough and thereby removed from the air. The droplets fall through the separation chamber under gravity and out of the separation chamber through the contaminated water outlet 22a. Suitably, the micro-organisms are entrapped in the water droplets along with other particulate matter and gases and discharged through the contaminated water outlet irrespective of whether they are dead or alive. Any micro-organisms that are still alive are immersed in the sanitized water in the tank 30 of the first column and are thereby killed. Meanwhile, the air flowing upward through the packed bed is cooled as well as being cleaned and then enters the second column where a similar process takes place after which the cleaned air is returned to the surgery through the supply air duct system 13. Advantageously, the second column removes any residual sanitizing agent which might remain in the air after passing through the first separation column. If necessary, a heater may be provided in the supply air duct to reheat the clean air to a suitable air conditioning temperature. It will be appreciated that the chilled water assists in the separation of pollutants from the air extracted from the surgery as well as cooling the air for return to the surgery with the result that the separation columns effectively act as a filtration and purification system as well as an air conditioning system. Additionally, the use of the two separation columns provides an opportunity for using water at different temperatures to achieve better separation of some gaseous pollutants from the extracted air and also the opportunity for maintaining the air above the packed bed in one column at a different temperature by the addition of heaters therein if desired. It should be noted that corresponding reference numbers are used in the drawings to refer to corresponding components in the pre-filter separation column.

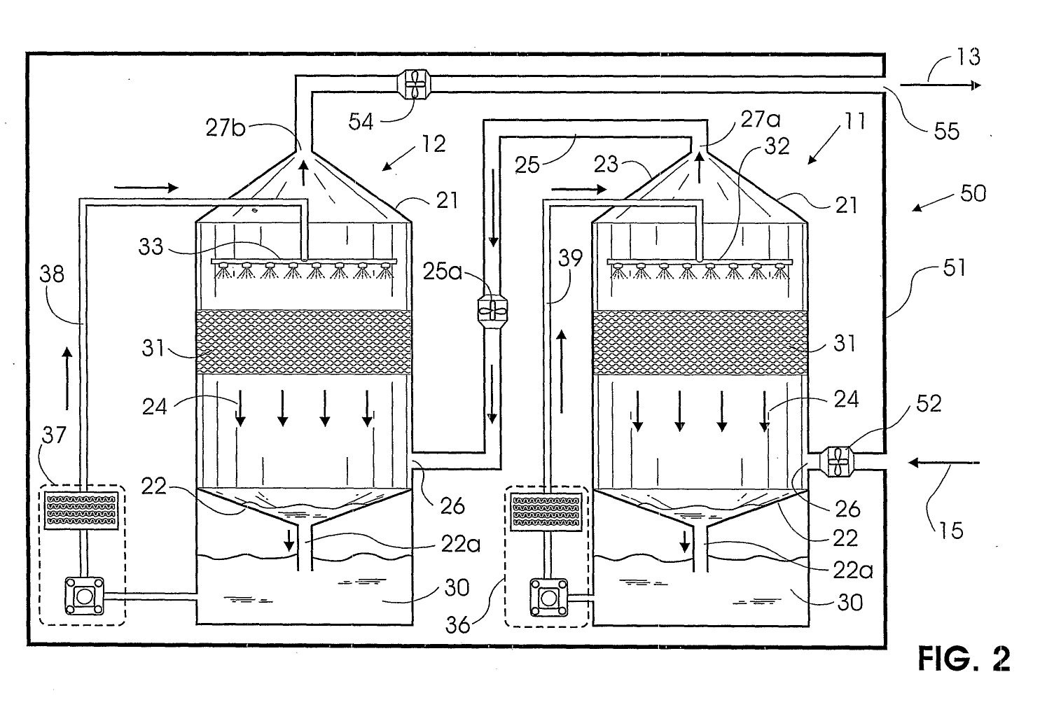

The system 50 illustrated in FIG. 2 operates in much the same manner as that illustrated in FIG. 1, the main difference being that the two separation columns 11 and 12 are housed in a box 50 which can operate as a self contained unit with the polluted air inlet 26 being ducted to the side wall of the box through a fan 52 to open at new inlet 53 and the clean air outlet being ducted to the side wall of the box through a fan 54 to a new clean air outlet 55.

The foregoing description has been given by way of illustrative example of the invention and many variations and modifications which will be apparent to persons skilled in the art may be made without departing from the spirit and scope of the invention as defined in the appended claims.

1. A filtration and purification system including: extraction means for extracting air from a confined space; filtration and purification means for filtering particulate from the extracted air and purifying the air by killing micro-organisms entrained in the extracted air.

2. A filtration and purification system including: extraction means for extracting air from a confined space; filtration means for filtering particulate from the extracted air; and purification means for purifying the air by killing micro-organisms which are entrained in the extracted air.

3. An air conditioning system including: extraction means for extracting air from a confined space; filtration and purification means for filtering particulate from the extracted air and purifying the air, the filtration and purification means including a separation column having an extracted air inlet for receiving extracted air therethrough, a packed bed at a predetermined first level above the extracted air inlet, water distribution means above the packed bed for distributing cold water onto the packed bed, a clean air outlet above the packed bed for discharging filtered and purified air to the confined space, and a contaminated water outlet.

4. An air conditioning system according to claim 3, including a tank containing sanitized water adapted to kill unwanted micro-organisms in liquid communication with the contaminated water outlet and a pump for supplying sanitized water from the tank to the water distribution means.

5. An air conditioning system according to claim 3, wherein the separation column is a first separation column and the system includes a second separation column downstream of the first separation column for filtering contaminants from the extracted air prior to entry to the second separation column, the first separation column having an extracted air inlet for receiving extracted air therethrough, a packed bed at a predetermined first level above the extracted air inlet, water distribution means above the packed bed for distributing sanitized cold water onto the packed bed, a clean air outlet above the packed bed for discharging air to the second separation column after passing through the packed bed therein and a contaminated water outlet, and a chiller for chilling the sanitized water prior to distribution through the water distribution means of the later separation column.

6. A method of sanitizing a room or a building, including: continuously extracting air from the room or building; passing the extracted air through filtration and purification means thereby filtering particulate from the extracted air and purifying the air by entrapping micro-organisms entrained therein in a sanitized liquid; and returning at least some of the extracted air to the room or building after filtration and purification by the filtration and purification means.

7. A method according to claim 6, wherein the filtration and purification means includes a separation column having an extracted air inlet for receiving the extracted air therethrough, a packed bed at a predetermined first level above the extracted air inlet and water distribution means above the packed bed for distributing cold water onto the packed bed and the extracted air is passed upwards through the packed bed while sanitized water is distributed onto the packed bed and allowed to flow downwards therethrough to entrap the micro-organisms.

8. A method according to claim 7, wherein the sanitized water distributed onto the packed bed is at a temperature of less than about 25 degrees Celsius.

9. A method according to claim 7, wherein the sanitized water distributed onto the packed bed is at a temperature of less than about 5 degrees Celsius.

10. A method according to claim 7, wherein the sanitized water distributed onto the packed bed is at a temperature of less than about 2 degrees Celsius.

11. A method according to claim 7, wherein the water distribution means provides a continuous spray across substantially the whole upper surface of the packed bed at a flow rate sufficient to prevent the growth of micro-organisms in the packed bed.

12. A method according to claim 6, wherein the air is reheated after passing through the packed bed of the separation column to achieve a suitable air conditioning temperature.

13. A method according to claim 7, wherein the separation column is a first separation column and the system includes a second separation column downstream of the first separation column, the second separation column has a purified air inlet for receiving air therethrough from the first separation column, a packed bed at a predetermined first level above the purified air inlet, water distribution means above the packed bed for distributing cold water onto the packed bed, a clean air outlet above the packed bed for discharging air to the after passing through the packed bed and a contaminated water outlet, and wherein the water distribution means of the second separation column sprays unsanitized water onto the packed bed of that separation column.

14. A method according to claim 7, wherein the system includes a tank to which the contaminated water flows from the contaminated water outlet of the first separation column and control means for maintaining the level of sanitizing agent in the water at a desired predetermined level.

15. A method according to claim 14, wherein the control means also controls the removal of contaminated water from the tank and the addition of fresh water thereto in order to maintain the level of contaminants in the water below a predetermined maximum desired level.

16. (canceled)

17. (canceled)

18. (canceled)
